(19) United States
(12) Patent Application Publication
(10) Pub. No.: US 2025/0009715 A1
(43) Pub. Date: Jan. 9, 2025

(54) PHARMACEUTICAL COMBINATIONS FOR TREATING CANCER

(71) Applicant: Purdue Pharmaceutical Products L.P., Stamford, CT (US)

(72) Inventors: Thomas Jorg Mehrling, Hunenberg (CH); Enrique Maria Ocio, Salamanca (ES)

(73) Assignee: Purdue Pharmaceutical Products L.P., Stamford, CT (US)

(21) Appl. No.: 18/743,233

(22) Filed: Jun. 14, 2024

Related U.S. Application Data
(63) Continuation of application No. 18/086,958, filed on Dec. 22, 2022, now Pat. No. 12,048,688.
Application No. 18/086,958 is a continuation of application No. 17/212,765, filed on Mar. 25, 2021, now Pat. No. 11,559,516.
Application No. 17/212,765 is a continuation of application No. 16/517,936, filed on Jul. 22, 2019, now abandoned.
Application No. 16/517,936 is a continuation of application No. 15/985,097, filed on May 21, 2018, now Pat. No. 10,406,138.
Application No. 15/985,097 is a continuation of application No. 15/314,162, filed on Nov. 28, 2016, now abandoned.

(30) Foreign Application Priority Data
May 28, 2014 (GB) 1409471.8

(51) Int. Cl. A61K 31/4184; A61K 31/407; A61K 31/427; A61K 31/502; A61K 31/5377; A61K 31/55; A61K 31/573; A61K 31/58; A61K 31/69; A61K 38/05; A61K 38/06; A61K 39/395; A61K 45/06

(52) U.S. Cl. CPC A61K 31/4184; A61K 31/407; A61K 31/427; A61K 31/502; A61K 31/5377; A61K 31/55; A61K 31/573; A61K 31/58; A61K 31/69; A61K 38/05; A61K 38/06; A61K 39/3955; A61K 45/06

(57) ABSTRACT

The present invention is directed to a combination comprising a proteasome inhibitor and a compound of formula I or a pharmaceutically acceptable salt thereof:

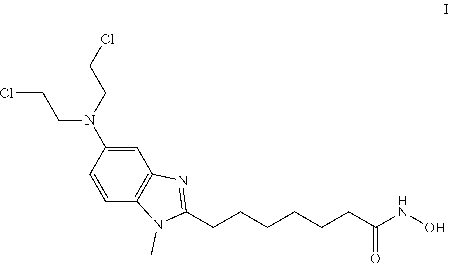 [SMILES: Cn1c(CCCCCCC(=O)NO)nc2cc(N(CCCl)CCCl)ccc21]

to a pharmaceutical composition and to a kit both comprising said combination, to the combination, composition or kit for use in the treatment of cancer, and to a method of treatment of cancer in a patient in need thereof comprising administering to said patient an effective amount of said combination, composition or kit.

### CROSS-REFERENCE TO RELATED APPLICATIONS

This application is a continuation application of U.S. patent application Ser. No. 18/086,958, filed Dec. 22, 2022, which is a continuation application of U.S. patent application Ser. No. 17/212,765, filed Mar. 25, 2021, now U.S. Pat. No. 11,559,516, which is a continuation application of U.S. patent application Ser. No. 16/517,936, filed on Jul. 22, 2019, now abandoned, which is a continuation application of U.S. patent application Ser. No. 15/985,097, filed on May 21, 2018, now U.S. Pat. No. 10,406,138, which is a continuation application of U.S. patent application Ser. No. 15/314,162, filed on Nov. 28, 2016, now abandoned, which is a U.S. national stage filing under 35 U.S.C. § 371(c) of International Application No. PCT/EP2015/061571, filed on May 26, 2015, which claims foreign priority of U.K. Patent Application No. 1409471.8, filed on May 28, 2014. The entire contents of each of the aforementioned applications, including original claims and drawings, are incorporated herein by reference.

### TECHNICAL FIELD

The present invention relates to combinations and compositions that are of use in the treatment of cancer, for example in the treatment of breast cancer or of hematologic cancers such as multiple myeloma, lymphoma or leukemia.

### BACKGROUND TO THE INVENTION

Cancer is one of the most life threatening diseases. Cancer is a condition in which cells in a part of the body experience out-of-control growth. According to latest data from American Cancer Society, it is estimated there will be 1.67 million new cases of cancer in USA in 2014. Cancer is the second leading cause of death in the United States (second only to heart disease) and will claim more than 585,000 lives in 2014. In fact, it is estimated that 50% of all men and 33% of all women living in the United States will develop some type of cancer in their lifetime. Therefore cancer constitutes a major public health burden and represents a significant cost in the United States. These figures are reflected elsewhere across most countries globally, although the types of cancer and relative proportions of the population developing the cancers vary depending upon many different factors such including genetics and diet.

For decades surgery, chemotherapy, and radiation were the established treatments for various cancers. Patients usually receive a combination of these treatments depending upon the type and extent of their disease. But chemotherapy is the most important option for cancer patients when surgical treatment (i.e. the removal of diseased tissue) is impossible. While surgery is sometimes effective in removing tumors located at certain sites, for example, in the breast, colon, and skin, it cannot be used in the treatment of tumors located in other areas, such as the backbone, nor in the treatment of disseminated hematologic cancers include cancers of the blood and blood-forming tissues (such as the bone marrow). They include multiple myeloma, lymphoma and leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer. One of the main causes of failure in this treatment of cancer is the development of drug resistance by the cancer cells, a serious problem that may lead to recurrence of disease or even death. Thus, more effective cancer treatments are needed.

Multiple myeloma is a significant and growing problem. It is a cancer arising from plasma cells. Normal plasma cells produce immunoglobulins to fight infection. In myeloma, the plasma cells become abnormal, multiply uncontrollably and release only one type of anttibody—known as paraprotein—which has no useful function. It tends to accumulate in the bone marrow and circulate in the blood and can be detected in the urine as well. It affects multiple sites in the body (hence ‘multiple’ myeloma) where bone marrow is normally active in adults. The main forms of multiple myeloma (or myeloma as it is also referred to) are active myeloma, plasmacytoma, light chain myeloma and non-secretory myeloma. The number of new cases of myeloma in the US in 2011 was 6.1 per 100,000 men and women per year and the percentage survival rate beyond five years was 45%. It is estimated that the number of new cases in the US in 2014 will be over 24,000 (1.4% of all cancer cases), while the number of deaths in 2014 will be just over 11,000 (1.9% of all cancer cases).

In WO-A-2010/085377, the compound of formula I was shown to have excellent in vitro activity against multiple myeloma cell lines, with activities in the range of ×35-100 greater than the activity shown by bendamustin.

Leukemia is a type of cancer of the blood or bone marrow characterized by an abnormal increase of immature white blood cells called “blasts”. Instead of producing normal, functioning white blood cells to fight infection the body produces large numbers of these non-functional blasts. Leukemia is a broad term covering a spectrum of diseases. In turn, it is part of the even broader group of diseases affecting the blood, bone marrow and lymphoid system, which are all known as hematological neoplasms. The most common forms are acute lymphoblastic leukemia (ALL), chronic lymphocytic leukemia (CLL), acute myeloid leukemia (AML) and chronic myeloid leukemia (CML), with less common forms including hairy cell leukemia (HCL), T-cell prolymphocytic leukemia (T-PLL), large granular lymphocytic leukemia and T-cell acute lymphoblastic leukemia. It is estimated that the number of new cases in the United States in 2014 will be over 52,000 (3.1% of all new cancers in the US) with over 24,000 deaths (4.1% of all cancer deaths in the US). The percentage survival rate over five years is currently 57.2%, a figure significantly lower than for many other cancers, with the survival rate over five years for acute myeloid leukemia being particularly low at only 20%.

Lymphoma is a cancer of the lymphatic system. There are two main types of lymphoma, namely Hodgkin lymphoma and non Hodgkin lymphoma.

Non Hodgkin lymphoma is the more common form of lymphoma. The lymphatic system runs throughout the body, and it is therefore possible to find non Hodgkin lymphoma in almost all parts of the body. In patients with non Hodgkin lymphoma, some of their white blood cells (lymphocytes) divide abnormally. They do not have any resting time like normal cells and they start to divide continuously, so too many are produced. They do not naturally die off as they usually do. These cells start to divide before they are fully mature and therefore cannot fight infection as normal white blood cells do. All the abnormal lymphocytes start to collect in the lymph nodes or other places such as the bone marrow or spleen. They can then grow into tumours and begin to cause problems within the lymphatic system or the organ in which they are growing. For example, if a lymphoma starts in the thyroid gland it can affect the normal production of thyroid hormones. There are many different types of non Hodgkin lymphoma. They can be classified in several different ways. One way is by the type of cell affected. In non Hodgkin lymphoma two types of lymphocyte can be affected—B cells and T cells. This is classified as B cell lymphoma or a T cell lymphoma. Most people with non Hodgkin lymphoma have B cell lymphomas. T cell lymphomas are more common in teenagers and young adults.

The cells of Hodgkin lymphoma have a particular appearance under the microscope. These cells are called Reed Sternberg cells. Non Hodgkin lymphomas do not have Reed Sternberg cells. It is important for doctors to be able to tell the difference between Hodgkin lymphoma and non Hodgkin lymphoma cells as they are two different diseases. In Hodgkin lymphoma, it is cells in the lymph nodes that have become cancerous.

The % survival rate over 5 years in 2009 for patients with non Hodgkin lymphoma was 63%, while the survival rate for those with Hodgkin lymphoma over the same period was 83%.

Breast cancer is a cancer that forms in tissues of the breast. The most common type of breast cancer is ductal carcinoma, which begins in the lining of the milk ducts (thin tubes that carry milk from the lobules of the breast to the nipple). Another type of breast cancer is lobular carcinoma, which begins in the lobules (milk glands) of the breast. Breast cancers can be classified into sub-groups as claudin-low tumors, basal-like tumors, human epidermal growth factor receptor 2 (HER2) positive tumors, luminal A tumors and luminal B tumors. Invasive breast cancer is breast cancer that has spread from where it began in the breast ducts or lobules to surrounding normal tissue. Breast cancer occurs in both men and women, although male breast cancer is rare. In 2014, it is estimated that there will be nearly 233,00 new cases in women and 2,400 in men, with 40,00 female deaths and just over 400 male deaths.

Approximately 15 out of every 100 women with breast cancer have triple-negative breast cancer, i.e. are estrogen negative, are progesterone negative and are HER2 negative. Recurrent triple-negative breast cancer is a condition of high unmet medical need, due to its aggressive biology, fast development of drug resistance and lack of molecular targets. Until now, chemotherapy remains the standard of care for advanced triple-negative breast cancer with a poor median overall survival.

In WO-A-2010/085377, the compound of formula I below is disclosed. It is a first-in-class dual-functional alkylating-HDACi fusion molecule which potently inhibits the HDAC pathway.

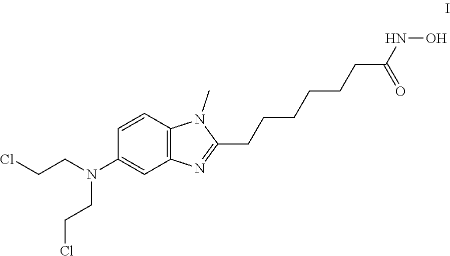 [SMILES: Cn1c(CCCCCCC(=O)NO)nc2cc(N(CCCl)CCCl)ccc21]

Biological assays showed that the compound of formula I potently inhibits HDAC enzyme (HDAC1 IC₅₀ of 9 nM) and it has been shown to have excellent in vitro activity against multiple myeloma cell lines.

There is a need for more effective cancer treatments, including the treatment of breast cancer and of hematologic cancers such as multiple myeloma, lymphoma or leukemia. Currently, these conditions affect many people and the medium to long-term prognosis is not good for many of these conditions.

### SUMMARY OF THE INVENTION

In a first aspect of the present invention there is provided a combination comprising a proteasome inhibitor and a compound of formula I or a pharmaceutically acceptable salt thereof:

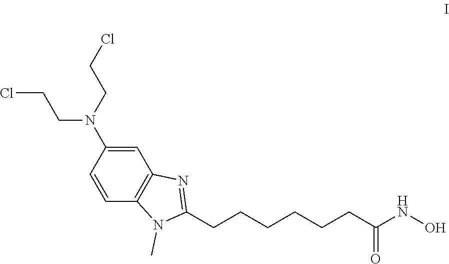 [SMILES: Cn1c(CCCCCCC(=O)NO)nc2cc(N(CCCl)CCCl)ccc21]

It has surprisingly been discovered that combinations of a compound of formula I or a pharmaceutically acceptable salt thereof and a proteasome inhibitor such as carfilzomib or bortezomib are particularly effective in the treatment of cancers including hematologic cancers such as multiple myeloma, lymphoma and leukemia, and breast cancer, such that they are highly promising in efforts to address the problem of finding more effective treatments for cancer. The combinations may optionally further comprise a glucocorticoid such as dexamethasone. These further combinations are also particularly effective in the treatment of cancer.

In a second aspect of the present invention, there is provided a pharmaceutical composition comprising a pharmaceutically acceptable diluent or carrier and a combination according to the first aspect of the invention.

In a third aspect of the present invention, there is provided a kit comprising a combination according to the first aspect of the present invention and, optionally, instructions for treating a patient.

In a fourth aspect of the present invention, there is provided a combination, composition or kit according to the first, second or third aspect of the present invention for use in the treatment of cancer.

In a fifth aspect of the present invention, there is provided a method of treating cancer in a patient in need thereof comprising administering to said patient a combination, composition or kit according to the first, second or third aspect of the present invention.

In a sixth aspect of the present invention, there is provided a compound of formula (I) or a pharmaceutically acceptable salt thereof for use in the treatment of relapsed/refractory multiple myeloma. In one embodiment, the compound of formula (I) or the pharmaceutically acceptable salt thereof is for use in the treatment of relapsed/refractory multiple myeloma in combination with a proteasome inhibitor and optionally further in combination with a glucocorticoid.

In a seventh aspect of the present invention, there is provided a method of treatment of relapsed/refractory multiple myeloma in a patient in need thereof comprising administering to said patient a compound of formula (I) or the pharmaceutically acceptable salt thereof. In one embodiment, the compound of formula (I) or the pharmaceutically acceptable salt thereof is administered in combination with a proteasome inhibitor and may further optionally be administered in combination with a glucocorticoid as well.

### DESCRIPTION OF THE DRAWINGS

FIG. 1 is a plot of the % surviving in vitro MM1S multiple myeloma cells as a % of control versus concentration for different tested compounds after 48 hours incubation, for single compounds and as combinations (double and triple);

FIG. 2 is a plot of the % surviving in vitro MM1S multiple myeloma cells as a % of control versus concentration for different tested compounds after 72 hours incubation, for single compounds and as combinations (double and triple);

FIG. 3 is a plot of tumour growth (mm³) against the number of days of study for different tested compounds for CB17-SCID mice subcutaneously inoculated into the right flank with 3×10⁶ MM1S cells, for single compounds and as combinations;

FIG. 4 is a plot of the % surviving in vitro RPMI8226 multiple myeloma cells as a % of control versus concentration for different tested compounds after 48 hours incubation, for single compounds and as combinations (double);

FIG. 5 is a plot of the % surviving in vitro 2013 Oct. 16 MTS AMO abzb multiple myeloma cells as a % of control versus concentration for different tested compounds after 48 hours incubation, for single compounds and as combinations (double);

FIG. 6 is a plot of the % surviving in vitro 2014 Jan. 15 MTS Jeko mantle cell lymphoma cells as a % of control versus concentration for different tested compounds after 48 hours incubation, for single compounds and as combinations (double);

FIG. 7 is a plot of the % surviving in vitro 2014 Jan. 15 MTS Granta mantle cell lymphoma cells as a % of control versus concentration for different tested compounds after 48 hours incubation, for single compounds and as combinations (double);

FIG. 8 is a plot of the % surviving in vitro 2014 Feb. 21 MTS MTS MDA-MB468 basal like breast cancer cells as a % of control versus concentration for different tested compounds after 48 hours incubation, for single compounds and as combinations (double);

FIG. 9 is a plot of the % surviving in vitro MTS HL-60 promyelocytic leukemia cells as a % of control versus concentration for different tested compounds after 48 hours incubation, for single compounds and as combinations (double);

FIG. 10 is a plot of the % surviving in vitro MTS U937 acute myeloid leukemia cells as a % of control versus concentration for different tested compounds after 48 hours incubation, for single compounds and as combinations (double);

FIG. 11 is a plot of the % surviving in vitro BJAB (germinal center line) B cell lymphoma cells as a % of control versus concentration for different tested compounds after 48 hours incubation, for single compounds and as combinations (double);

FIG. 12 is a plot of the % surviving in vitro OciLy3 (ABC-type) B cell lymphoma cells as a % of control versus concentration for different tested compounds after 48 hours incubation, for single compounds and as combinations (double);

FIG. 13 is a plot of the % surviving in vitro TMD8 (ABC-type) B cell lymphoma cells as a % of control versus concentration for different tested compounds after 48 hours incubation, for single compounds and as combinations (double);

FIG. 14 is a plot of the % surviving in vitro BT-549 triple negative breast cancer cells as a % of control versus concentration for different tested compounds after 48 hours incubation, for single compounds and as combinations (double); and

FIG. 15 is a plot of % surviving fraction of in vitro T98G, U251MG and U87MG glioblastoma cell lines against dose of radiotherapy (Gy) in combination with two different concentrations of the compound of formula I (EDO-S101) against a control with radiotherapy alone.

### DETAILED DESCRIPTION OF THE INVENTION

In the present application, a number of general terms and phrases are used, which should be interpreted as follows.

“Animal” includes humans, non-human mammals (e.g., dogs, cats, rabbits, cattle, horses, sheep, goats, swine, deer, and the like) and non-mammals (e.g., birds, and the like).

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

It has surprisingly been discovered that combinations of a compound of formula I or a pharmaceutically acceptable salt thereof and a proteasome inhibitor such as carfilzomib or bortezomib are particularly effective in the treatment of cancers including hematologic cancers such as multiple myeloma, leukemia and lymphoma, and breast cancer such that they are highly promising in efforts to address the problem of finding more effective treatments for cancer. The combinations may optionally further comprise a glucocorticoid such as dexamethasone. These further combinations are also particularly effective in the treatment of cancer.

In the combination of the present invention, the pharmaceutically acceptable salt of the compound of formula I may preferably be the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate or acetate, and more preferably the acetate.

In the combination of the present invention, the proteasome inhibitor may preferably be selected from the group consisting of bortezomib, carfilzomib, marizomib, delanzomib (CEP-18770), oprozomib (ONX 0912), ixazomib (MLN-9708) and LU-102, or a pharmaceutically acceptable salt thereof. Particularly preferably, the proteasome inhibitor may be selected from bortezomib, carfilzomib and LU-102.

The structures of these proteasome inhibitors are as follows:

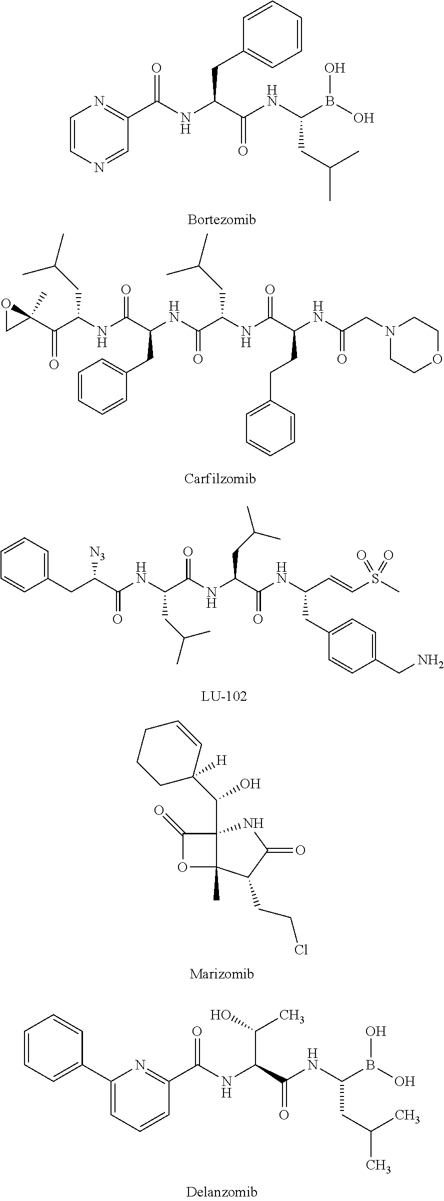 [SMILES: CC(C)C[C@H](NC(=O)[C@H](Cc1ccccc1)NC(=O)c1cnccn1)B(O)O ; CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1 ; CC(C)C[C@H](NC(=O)[C@H](Cc1ccccc1)N=[N+]=[N-])C(=O)N[C@@H](CC(C)C)C(=O)N[C@H](/C=C/S(C)(=O)=O)Cc1ccc(CN)cc1 ; C[C@@]12OC(=O)[C@]1([C@@H](O)[C@@H]1C=CCCC1)NC(=O)[C@@H]2CCCl ; CC(C)C[C@H](NC(=O)[C@@H](NC(=O)c1cccc(-c2ccccc2)n1)[C@@H](C)O)B(O)O]

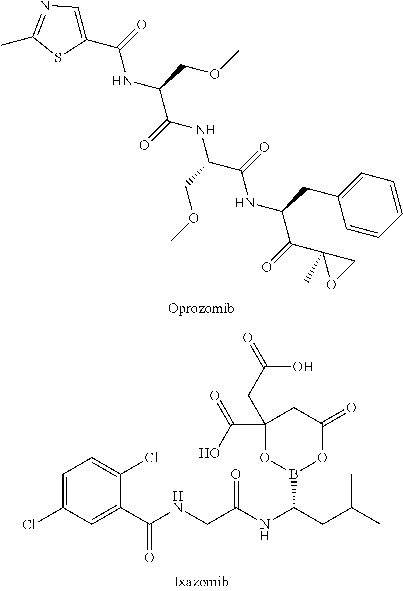 [SMILES: COC[C@H](NC(=O)c1cnc(C)s1)C(=O)N[C@@H](COC)C(=O)N[C@@H](Cc1ccccc1)C(=O)[C@@]1(C)CO1 ; CC(C)C[C@H](NC(=O)CNC(=O)c1cc(Cl)ccc1Cl)B1OC(=O)CC(CC(=O)O)(C(=O)O)O1]

The combination of the present invention may further comprise a glucocorticoid. In this embodiment of the combination of the present invention, the glucocorticoid may preferably be selected from the group consisting of dexamethasone, fluocinolone acetonide and prednisone, and it is most preferably dexamethasone.

In one further preferred combination of the present invention comprising a compound of formula I or a pharmaceutically acceptable salt thereof, a proteasome inhibitor and optionally a glucocorticoid, said combination may further comprise one or more additional pharmaceutically active agents. Particularly suitable pharmaceutically active agents are anti-tumor agents having a different mode of action to the compound of formula I or a pharmaceutically acceptable salt thereof, the proteasome inhibitor and the glucocorticoid, e.g. alkylating agents such as nitrosureas, ethylenimines, alkylsulfonates, hydrazines and triazines, and platinum based agents; plant alkaloids, taxanes, vinca alkaloids; anti-tumor antibiotics such as chromomycins, anthracyclines, and miscellaneous antibiotics such as Mitomycin and Bleomycin; anti-metabolites such as folic acid antagonists, pyrimidine antagonists, purine antagonists and adenosine deaminase inhibitors; topoisomerase inhibitors such as topoisomerase I inhibitors, topoisomerase II inhibitors, miscellaneous anti-neoplastics such as ribonucleotide reductase inhibitors, adrenocortical steroid inhibitor, anti-microtubule agents, and retinoids; protein kinases; heat shock proteins, poly-ADP (adenosine diphosphate)-ribose polymerase (PARP), hypoxia-inducible factors (HIF), proteasome, Wnt/Hedgehog/Notch signaling proteins, TNF-alpha, matrix metalloproteinase, farnesyl transferase, apoptosis pathway, histone deacetylases (HDAC), histone acetyltransferases (HAT), and methyltransferase; hormonal therapies, vascular disrupting agent, gene therapy, RNAi cancer therapy, chemoprotective agents, antibody conjugate, cancer immunotherapy such as Interleukin-2, cancer vaccines or monoclonal antibodies; and preferably DNA damaging agents, anti-metabolites, topoisomerase inhibitors, anti-microtubule agents, EGFR inhibitors, HER2 inhibitors, VEGFR2 inhibitors, BRAF inhibitors, Bcr-Abl inhibitors, PDGFR inhibitors, ALK inhibitors, PLK inhibitors, MET inhibitors, epigenetic agents, HSP90 inhibitors, PARP inhibitors, CHK inhibitors, aromatase inhibitor, estrogen receptor antagonist, and antibodies targeting VEGF, HER2, EGFR, CD50, CD20, CD30, CD33, etc.

In one preferred embodiment of the combination of the present invention, the proteasome inhibitor, the compound of formula I or a pharmaceutically acceptable salt thereof and, if present, the glucocorticoid are adapted for administration concurrently, sequentially or separately. Preferably, the proteasome inhibitor, the compound of formula I or a pharmaceutically acceptable salt thereof and, if present, the glucocorticoid are adapted for administration concurrently.

In one preferred embodiment of the combination of the present invention, the proteasome inhibitor is selected from bortezomib, carfilzomib and LU-102 and the compound of formula I or a pharmaceutically acceptable salt thereof is

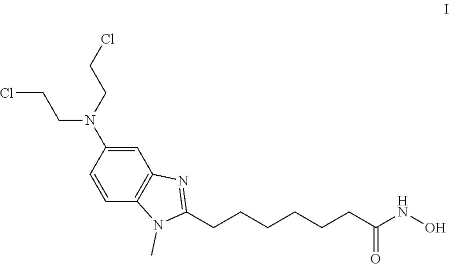 [SMILES: Cn1c(CCCCCCC(=O)NO)nc2cc(N(CCCl)CCCl)ccc21]

or the acetate salt thereof. In one embodiment of this combination, the combination may further comprise a glucocorticoid wherein said glucocorticoid is dexamethasone.

In one preferred embodiment of the combination of the the present invention, the molar ratio of proteasome inhibitor to compound of formula I or a pharmaceutically acceptable salt thereof in said combination is from 1:1000 to 1000:1. Preferably, the molar ratio of proteasome inhibitor to compound of formula I or a pharmaceutically acceptable salt thereof in said combination is from 1:1000 to 10:1, more preferably from 1:800 to 1:200 or from 1:5 to 1:0.5, and most preferably it is from 1:700 to 1:400 or from 1:3 to 1:0.5, e.g. 1:1000, 1:900, 1:800, 1:700, 1:600, 1:500, 1:400, 1:10, 1:5, 1:4, 1:3, 1:2, 1:1 or 1:0.5.

One particularly preferred combination of present invention comprises the compound of formula I or the acetate salt thereof and a proteasome inhibitor selected from bortezomib and carfilzomib, wherein the molar ratio of the proteasome inhibitor selected from bortezomib and carfilzomib to the compound of formula I or a pharmaceutically acceptable salt thereof in said combination is from 1:700 to 1:400, e.g. 1:700, 1:600, 1:500 or 1:400. Another particularly preferred combination of the first aspect of the present invention comprises the compound of formula I or the acetate salt thereof and a proteasome inhibitor selected from LU-102, wherein the molar ratio of LU-102 to the compound of formula I or a pharmaceutically acceptable salt thereof in said combination is from 1:3 to 1:0.5, e.g. 1:3, 1:2, 1:1 or 1:0.5.

It has been surprisingly found that combinations comprising a proteasome inhibitor and a compound of formula I or a pharmaceutically acceptable salt thereof are synergistic combinations. In other words, the potency of the combinations was measured with the Calcusyn software (biosoft, Ferguson, MO, USA), which is based on the Chou Talay method (Chou et al., Adv. Enzyme Regul., 22, 27-55 (1984)), that calculates a combination index (CI) with the following interpretation:
CI 1>1: antagonist effect, CI=1: additive effect and CI<1 synergistic effect.

It was found in the present work that for many of the dual combinations of the invention comprising a proteasome inhibitor and a compound of formula I or a pharmaceutically acceptable salt, CI has been found to be less than 1, indicating synergy.

Another preferred embodiment of the combination of the present invention further comprises a glucocorticoid in addition to the proteasome inhibitor and the compound of formula I or a pharmaceutically acceptable salt thereof, wherein the molar ratio of proteasome inhibitor to the compound of formula I or a pharmaceutically acceptable salt thereof to the glucocorticoid in said combination is from 1:1000:20 to 1000:1:20. Preferably, the molar ratio of proteasome inhibitor to the compound of formula I or a pharmaceutically acceptable salt thereof to the glucocorticoid in said combination is from 1:1000:10 to 1:100:2. Preferably, the molar ratio of proteasome inhibitor to the compound of formula I or a pharmaceutically acceptable salt thereof to the glucocorticoid used in said combination is from 1:1000:5 to 1:200:2, more preferably 1:700:4 to 1:400:3, e.g. 1:1000:5, 1:900:5, 1:800:4, 1:700:4, 1:600:4, 1:500:3 or 1:400:3.

One particularly preferred combination of the the present invention comprises a proteasome inhibitor selected from bortezomib and carfilzomib, a compound of formula I or the acetate salt thereof and dexamethasone, wherein the molar ratio of the proteasome inhibitor selected from bortezomib and carfilzomib to the compound of formula I or the acetate salt thereof to dexamethasone in said combination is from 1:700:4 to 1:400:3, e.g. 1:700:4, 1:700:3, 1:600:4, 1:600:3, 1:500:3 or 1:400:3. Another particularly preferred combination of the first aspect of the present invention comprises a proteasome inhibitor selected from LU-102, the compound of formula I or the acetate salt thereof and dexamethasone, wherein the molar ratio of LU-102 to the compound of formula I or the acetate salt thereof to dexamethasone in said combination is from 1:3:4 to 1:0.5:3, e.g. 1:3:4, 1:3:3, 1:2:4, 1:2:3, 1:1,:4, 1:1:3 or 1:0.5:3.

It has also been surprisingly discovered that many of the triple combinations of the present invention comprising a proteasome inhibitor, a compound of formula I or a pharmaceutically acceptable salt thereof and a glucocorticoid are also synergistic combinations, i.e. the combination index CI has been found to be less than 1.

The pharmaceutical composition according to the second aspect of the present invention comprises a pharmaceutically acceptable diluent or carrier and a combination according to the first aspect of the present invention. Preferred compositions of the second invention include those comprising the preferred combinations of the present invention as described and exemplified above. The pharmaceutically acceptable diluent or carrier of the pharmaceutical composition according to the second aspect of the present can be any suitable dispersant, excipient, adjuvant, or other material which acts as a carrier for the active agents of the combination of the present invention and which does not interfere with the active agents present in said combination. Examples of typical pharmaceutically acceptable carriers and diluents may be found in “Remington's Pharmaceutical Sciences” by E. W. Martin and these include water, saline, dextrose solution, serum solution, Ringer's solution, polyethylene glycol (e.g PEG400), a surfactant (e.g Cremophor), a cyclopolysaccharide (e.g hydroxypropyl-β-cyclodextrin or sulfobutyl ether β-cyclodextrins), a polymer, a liposome, a micelle, a nanosphere, etc.

In the third aspect of the present invention, there is provided a kit comprising a combination according to the first aspect of the present invention and, optionally, instructions for treating a patient. Typically, a kit can comprise a compound of formula I or pharmaceutically acceptable salt thereof, a proteasome inhibitor, and a glucocorticoid together with instructions for treating a patient. Each active agent can be provided in a suitable container. The kit may further comprise a delivery system, e.g. for the compound of formula I or pharmaceutically acceptable salt thereof, the proteasome inhibitor or the glucocorticoid or any combination thereof.

The instructions may advise administering the proteasome inhibitor, the compound of formula I or a pharmaceutically acceptable salt thereof and, if present, the glucocorticoid of the combination concurrently, sequentially or separately according to variables such as the specific condition being treated, the state of that condition, the activity of the specific compounds employed; the specific combination employed; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts. Preferred kits according to the third aspect of the present invention include those comprising the preferred combinations of the present invention as described and exemplified above.

In the fourth aspect of the present invention, there is provided the combination, composition or kit according to the first, second or third aspect of the present invention for use in the treatment of cancer.

In the fifth aspect of the present invention, there is provided a method of treating cancer in a patient in need thereof comprising administering to said patient the combination, composition or kit according to the first, second or third aspect of the present invention.

It has been found that the combinations, compositions and kits of the present invention are highly active both in vitro and in vivo against a wide variety of tumour cell types. The anti-tumour activity shown by these double and triple combinations of the present invention, and by the combinations in the compositions and kits of the present invention is, in many cases, more than merely additive, showing combination indexes CI of significantly less than 1, indicating synergy for these combinations. This surprising finding is a further support for the particular effectiveness of the combinations, compositions and kits of the present invention in the treatment of cancer.

Examples of cancers which are treatable by the combinations, compositions and kits of the present invention include hematologic cancers such as multiple myeloma, lymphoma and leukemia, breast cancer, lung cancer, colorectal cancer, prostate cancer, testicular cancer, pancreatic cancer, liver cancer, stomach cancer, biliary tract cancer, esophageal cancer, gastrointestinal stromal tumor, cervical cancer, ovarian cancer, uterine cancer, renal cancer, melanoma, basal cell carcinoma, squamous cell carcinoma, bladder cancer, sarcoma, mesothelioma, thymoma, myelodysplastic syndrome, glioblastoma and myeloproliferative disease. In particular, the combinations, compositions and kits of the present invention are effective against hematologic cancer such as multiple myeloma, lymphoma and leukemia, and breast cancer.

In one embodiment of the combination, composition or kit for use in the treatment of a cancer according to the fourth aspect of the present invention or the method of treatment in accordance with the fifth aspect of the present invention, the cancer is selected from a hematologic cancer and breast cancer.

Where the combination, composition or kit of the present invention is for use in the treatment of a hematologic cancer, this may preferably be selected from multiple myeloma (e.g. active myeloma, plasmacytoma, light chain myeloma or non-secretory myeloma, with all forms being treatable in all phases including relapsed and refractory phases), lymphoma (e.g. Hodgkin lymphoma or non-Hodgkin lymphoma) and leukemia [acute lymphoblastic leukemia (ALL), chronic lymphocytic leukemia (CLL), acute myeloid leukemia (AML, including myeloblastic leukemia, acute promyelocytic leukemia, acute myelomonocytic leukemia, acute monocytic leukemia, acute erythroleukemia and acute megakaryotic leukemia, with all forms being treatable in all phases including relapsed and refractory phases), chronic myeloid leukemia (CML), hairy cell leukemia (HCL), T-cell prolymphocytic leukemia (T-PLL), large granular lymphocytic leukemia or T-cell acute lymphoblastic leukemia].

Where the combination, composition or kit of the present invention is for use in the treatment of breast cancer, the breast cancer may typically be selected from claudin-low tumors, basal-like tumors, human epidermal growth factor receptor 2 (HER2) positive tumors, luminal A tumors and luminal B tumors, and it is preferably a triple-negative breast cancer.

In one preferred embodiment of the combination, composition or kit for use in the treatment of cancer according to the present invention and the method of treatment of cancer according to the present invention, the proteasome inhibitor, the compound of formula I or a pharmaceutically acceptable salt thereof and, if present, the glucocorticoid are administered concurrently, sequentially or separately. More preferably, the proteasome inhibitor, the compound of formula I or a pharmaceutically acceptable salt thereof and, if present, the glucocorticoid are administered concurrently.

In the combination for use in the treatment of cancer and the method of treatment of cancer in accordance with the present invention, the compound of formula I or a pharmaceutically acceptable salt thereof is typically administered to the patient in need thereof at a dosage range of 10 to 100 mg/kg body weight patient, and preferably at a dosage range of 40 to 80 mg/kg body weight patient. Typically, the proteasome inhibitor is administered to the patient in need thereof at a dosage range of 0.01 to 0.3 mg/kg body weight patient, more preferably at a dosage range of 0.05 to 0.15 mg/kg body weight patient. Where a glucocorticoid is also administered in the combination, the glucocorticoid is typically administered at a dosage range of from 0.1 to 1 mg/kg body weight patient. Preferably, it is administered at a dosage range of from 0.3 to 0.5 mg/kg body weight patient.

The therapeutically effective amount of a combination, composition or kit according to the present invention is an amount of the combination, composition or kit which confers a therapeutic effect in accordance with the fourth and fifth aspects of the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of the combination, composition or kit according to the present invention is believed to be one wherein the compound of formula I or a salt thereof is included in the combination at a dosage range of from 10 to 100 mg/kg body weight patient (e.g. 40 to 80 mg/kg body weight such as 40, 50, 60, 70 or 80 mg/kg body weight), the proteasome inhibitor is included at a dosage range of from 0.01 to 0.3 mg/kg body weight patient (e.g. 0.1 to 1 mg/kg such as 0.1, 0.2, 0.3, 0.4 or 0.5 mg/kg body weight) and the glucocorticoid is included at a dosage range of from 0.03 to 1 mg/kg body weight patient (e.g. 0.3 to 0.5 mg/kg body weight patient, such as 0.3, 0.4 or 0.5 mg/kg body weight patient).

Effective doses will vary depending on route of administration, as well as the possibility of co-usage with other active agents. It will be understood, however, that the total daily usage of the combinations, compositions and kits of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The present invention is also directed to the use of a combination, composition or kit according to the first, second or third aspect of the present invention in the manufacture of a medicament for the treatment of cancer, e.g. for the treatment of a hematologic cancer or breast cancer.

Suitable examples of the administration form of the combination, composition or kit of the present invention include without limitation oral, topical, parenteral, sublingual, rectal, vaginal, ocular, and intranasal. Parenteral administration includes subcutaneous injections, intravenous, intramuscular, intrasternal injection or infusion techniques. Preferably, the combinations, compositions and kits are administered parenterally. Combinations and compositions of the invention can be formulated so as to allow a combination or composition of the present invention to be bioavailable upon administration of the combination or composition to an animal, preferably human. Compositions can take the form of one or more dosage units, where for example, a tablet can be a single dosage unit, and a container of a combination or composition of the present invention in aerosol form can hold a plurality of dosage units.

Preferably the combinations of the present invention are provided in the form of kits. Typically, a kit includes a proteasome inhibitor, a compound of formula I or a pharmaceutically acceptable salt thereof and, optionally, a glucocorticoid. In certain embodiments, a kit can include one or more delivery systems, e.g. the proteasome inhibitor, the compound of formula I or a pharmaceutically acceptable salt thereof and, optionally, a glucocorticoid, or any combination thereof, and directions for the use of the kit (e.g. instructions for treating a subject). These directions/instructions may advise administering the proteasome inhibitor, the compound of formula I or a pharmaceutically acceptable salt thereof and, if present, the glucocorticoid of the combination concurrently, sequentially or separately according to variables such as the specific condition being treated, the state of that condition, the activity of the specific compounds employed; the specific combination employed; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts.

The pharmaceutically acceptable diluent or carrier can be particulate, so that the compositions are, for example, in tablet or powder form. The carrier(s) can be liquid, with the combinations, compositions or kits being, for example, an oral syrup or injectable liquid. In addition, the carrier(s) can be gaseous, so as to provide an aerosol composition useful in, for example, inhalatory administration. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the combination, composition or kit of the present invention and the pharmaceutically acceptable carriers are sterile. Water is a preferred carrier when the combination or composition of the present invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

When intended for oral administration, the combination, composition or kit may be in solid or liquid form, where semi-solid, semi-liquid, suspension and gel forms are included within the forms considered herein as either solid or liquid.

As a solid composition for oral administration, the combination, composition or kit can be formulated into a powder, granule, compressed tablet, pill, capsule, chewing gum, wafer or the like form. Such a solid composition typically contains one or more inert diluents, either as a single tablet comprising all active agents or as a number of separate solid compositions, each comprising a single active agent of the combination of the present invention (in the case of the kit). In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When the combination or composition is in the form of a capsule (e. g. a gelatin capsule), it can contain, in addition to materials of the above type, a liquid carrier such as polyethylene glycol, cyclodextrin or a fatty oil.

The combination, composition or kit can be in the form of a liquid, e. g. an elixir, syrup, solution, emulsion or suspension. The liquid can be useful for oral administration or for delivery by injection. When intended for oral administration, a combination, composition or kit can comprise one or more of a sweetening agent, preservatives, dye/colorant and flavor enhancer. In a combination or composition for administration by injection, one or more of a surfactant, preservative, wetting agent, dispersing agent, suspending agent, buffer, stabilizer and isotonic agent can also be included. In the kit of the present invention, the liquid components comprising one or more of the active agents of the composition may either be combined prior to administration and administered concurrently or each active agent may be administered sequentially or separately.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, the present combinations, compositions and kits of the present invention are administered intravenously.

The liquid combinations, compositions and kits of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

For administration (e.g. intravenous) the combination, composition or kit may typically comprise the compound of formula I or a salt thereof at a dosage range of from 10 to 100 mg/kg body weight patient, the proteasome inhibitor at a dosage range of from 0.01 to 0.3 mg/kg body weight patient and the glucocorticoid at a dosage range of from 0.03 to 1 mg/kg body weight patient. More preferably, the combination, composition or kit may typically comprise the compound of formula I or a salt thereof at a dosage range of from 40 to 80 mg/kg body weight patient, the proteasome inhibitor at a dosage range of from 0.05 to 0.15 mg/kg body weight patient and the glucocorticoid at a dosage range of from 0.3 to 0.5 mg/kg body weight patient.

The combinations of the inventions may be formulated such that the proteasome inhibitor, the compound of formula I or a pharmaceutically acceptable salt thereof and, if present, the optional glucocorticoid of the combination are adapted for administration concurrently, sequentially or separately. Preferably, they are administered concurrently.

The combination, composition or kit of the present invention can be administered by any convenient route, for example by infusion or bolus injection, by absorption through epithelial or mucocutaneous linings.

In specific embodiments, it can be desirable to administer one or more combinations, compositions or kits of the present invention or combinations, compositions or kits locally to the area in need of treatment. In one embodiment, administration can be by direct injection at the site (or former site) of a cancer, tumor or neoplastic or pre-neoplastic tissue.

Pulmonary administration can also be employed, e. g. by use of an inhaler or nebulizer, and formulation with an aerosolizing agent, or via perfusion in a fluorocarbon or synthetic pulmonary surfactant. In certain embodiments, the combination, composition or kit of the present invention or compositions can be formulated as a suppository, with traditional binders and carriers such as triglycerides.

The present combination, composition or kit can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington's Pharmaceutical Sciences” by E. W. Martin.

The pharmaceutical combinations, compositions and kits can be prepared using methodology well known in the pharmaceutical art. For example, a composition intended to be administered by injection can be prepared by combining the components of a kit of the present invention with water so as to form a solution. A surfactant can be added to facilitate the formation of a homogeneous solution or suspension.

The combinations, compositions and kits of the present invention are particularly effective in the treatment of cancer.

The combinations of the present invention have been shown to have excellent activity against a wide variety of tumor cell types both in vitro and in vivo, making them particularly interesting for development for use in the treatment of cancer, e.g. hematologic cancer and breast cancer.

It has also discovered in the present work that the compound of formula I or a salt thereof can be administered in combination with radiotherapy in the treatment of glioblastoma. Both in vitro and in vivo studies showed that a combination of the compound of formula I or a salt thereof together with radiotherapy was far more effective than radiotherapy alone. There is a prior disclosure in WO 2013/113838 of data for the compound of formula I tested in the CNS Cancer (Glioma) cell lines SF-268, SF-295, SF-539, SNB-19, SNB-75 and U-251. These suggest activity for the compound of formula I against glioblastoma when used on its own.

### EXAMPLES

In the following examples, the compound having the following formula I is referred to as EDO-S101 (or EDO in the Figures):

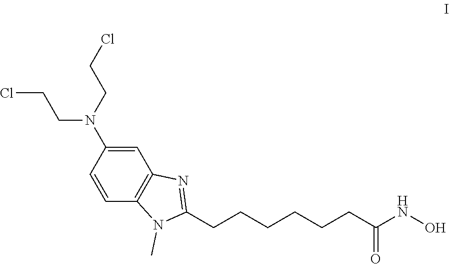 [SMILES: Cn1c(CCCCCCC(=O)NO)nc2cc(N(CCCl)CCCl)ccc21]

### Example 1 EDO-S101 Combinations In Vitro—Multiple Myeloma MM1S Cell Line

EDO-S101 was combined in vitro with bortezomib and dexamethasone in the multiple myeloma MM1S cell line kindly provided by Steven Rosen at Northwestern University, Chicago, IL, USA. Activity was measured by the MTT assay that is based on the metabolic bromide reduction from 3-(4,5-dimethylthiazol-2-yl)-2,5-diphenyltetrazole (MTT), which is produced by the mitochondrial enzyme succinate-dehydrogenase, turned to a blue-colored compound named formazan. The mitochondrial functionality of the treated cells is then determined. This method has been extensively used to measure cell proliferation and survival capacities. The remaining living cells are proportional to the amount of formazan produced.

In brief the methodology was as follows:
30,000 MM1S cells per well were plated into the 96-well microtiter plates.
EDO-S101 and PI dilutions were prepared in DMSO and dexamethasone in ethanol and added into the wells to the final concentrations indicated in the experiment.
Plates were incubated for 24-48-72 hours in the incubator at 37° C. in a humidified atmosphere in the presence of 5% CO₂/95% air.
After the incubation 10 μL of MTT solution were added in each well and incubated for 2 hours to allow formazan crystal formation.
100 μl of a mix solution with SDS plus HCl (10 μL of HCl for each 12 mL of SDS) was added to dissolve the formazan crystals.
Absorbance was read at 570 nm OD and use a reference wavelength of 650 nm.
Cell viability (percentage) was obtained as follows: % Viability=OD treated cells×100/OD control cells.
Each dose was tested in quadruplicate and each experiment was performed at least twice.

The concentrations for the different drugs were ratio constant for all the experiments. EDO-S101 at 500 nM, 1 μM, 2.5 μM; dexamethasone at 2.5 nM; 5 nM; 10 nM; and bortezomib at 0.75 nM, 1.5 nM, 3 nM.

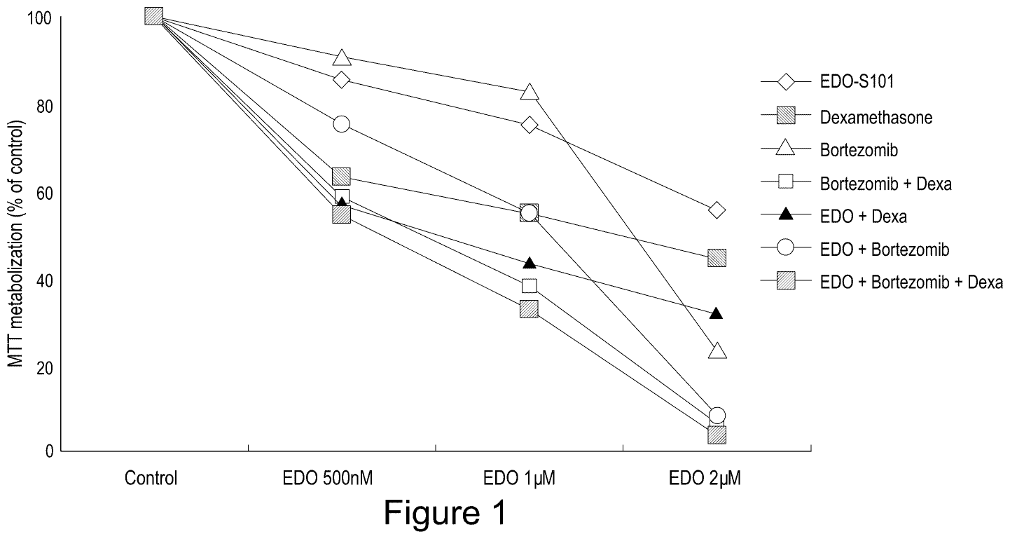

The results are as shown in Table 1 below and FIG. 1.

TABLE 1
48 H

| CI For experimental values | | | | |
|---|---|---|---|---|
| Dexa 48 h | | EDO 48 h | | |
| (nM) | | (nM) | Fa | CI |
| 2.5 | | 500 | 0.43453 | 0.851 |
| 5 | | 1000 | 0.56838 | 0.761 |
| 10 | | 2000 | 0.683802 | 0.765 |
| CI For experimental values | | | | |
| Bortz 48 h | | EDO 48 h | | |
| (nM) | | (nM) | Fa | CI |
| 0.75 | | 500 | 0.247333 | 1.087 |
| 1.5 | | 1000 | 0.452958 | 1.230 |
| 3 | | 2000 | 0.918526 | 0.627 |
| CI For experimental values | | DOBLE | | |
| Dexa 48 h | | Bortz 48 h | | |
| (nM) | | (nM) | Fa | CI |
| 2.5 | | 0.75 | 0.413191 | 1.105 |
| 5 | | 1.5 | 0.620757 | 0.879 |
| 10 | | 3 | 0.935984 | 0.494 |
| CI For experimental values | | | | |
| Dexa 48 h | Bortz 48 h | EDO 48 h | | |
| (nM) | (nM) | (nM) | Fa | CI |
| 2.5 | 0.75 | 150 | 0.455868 | 0.958 |
| 5 | 1.5 | 300 | 0.673133 | 0.789 |
| 10 | 3 | 600 | 0.962173 | 0.404 |

The potency of the combination was quantitated with the Calcusyn software (biosoft, Ferguson, MO, USA), which is based on the Chou Talay method (Chou et al., Adv. Enzyme Regul., 22, 27-55 (1984)), that calculates a combination index (CI) with the following interpretation:
CI 1>1: antagonist effect, CI=1: additive effect and CI<1 synergistic effect.

It can be seen from the FIG. 1 and from above that EDO-S101 shows synergy with both bortezomib and also shows synergy in a triple combination with bortezomib and dexamethasone.

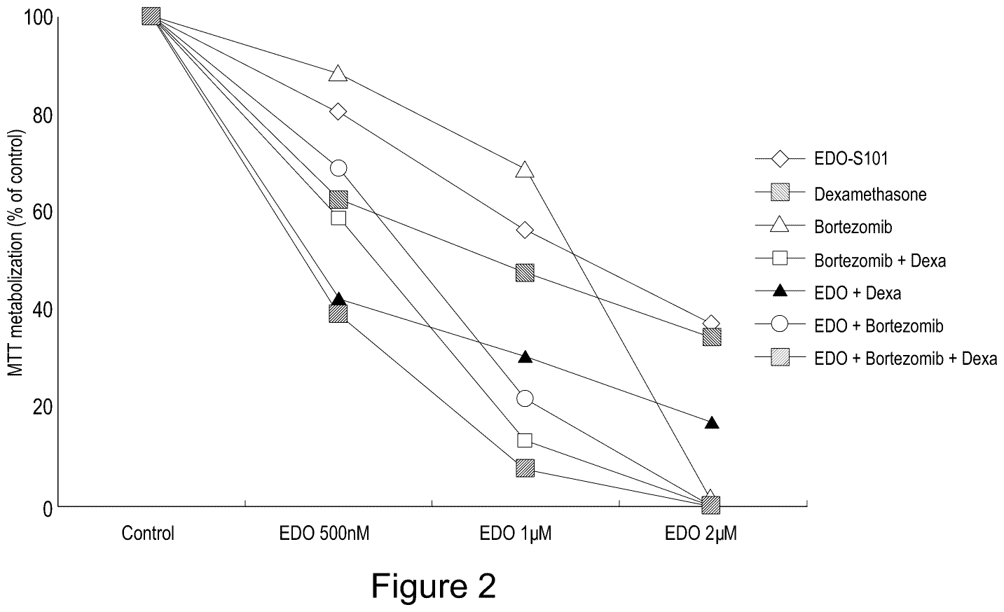

In a further experiment, the same constant dose of these drugs was incubated for 72 hours instead of 48 hours. The results are as shown in Table 2 below and FIG. 2

TABLE 2
72 H

| CI For experimental values | | | |
|---|---|---|---|
| DEXA | EDO | | |
| (nM) | (nM) | Fa | CI |
| 2.5 | 500 | 0.576413 | 0.682 |
| 5 | 1000 | 0.69365 | 0.836 |
| 10 | 2000 | 0.828332 | 0.829 |
| CI For experimental values | | | |
| BORTZ | EDO | | |
| (nM) | (nM) | Fa | CI |
| 0.75 | 500 | 0.310537 | 1.336 |
| 1.5 | 1000 | 0.780181 | 1.166 |
| 3 | 2000 | 0.999302 | 0.489 |
| CI For experimental values | | | |
| DEXA | BORTZ | | |
| (nM) | (nM) | Fa | CI |
| 2.5 | 0.75 | 0.411026 | 1.441 |
| 5 | 1.5 | 0.865318 | 0.876 |
| 10 | 3 | 1 | 0.017 |

| CI For experimental values | | | | |
|---|---|---|---|---|
| DEXA | BORTZ | EDO | | |
| (nM) | (nM) | (nM) | Fa | CI |
| 2.5 | 0.75 | 500 | 0.607118 | 1.115 |
| 5 | 1.5 | 1000 | 0.923936 | 0.845 |
| 10 | 3 | 2000 | 1 | 0.017 |

Again, it can be seen from FIG. 2 and the above results in Table 2 that EDO-S101 shows synergy with bortezomib and also shows synergy in a triple combination with bortezomib and dexamethasone.

### Example 2 EDO-S101 Combinations In Vivo Against a Xenograft of Subcutaneous Plasmacytoma

CB17-SCID mice (obtained from The Jackson Laboratory, Bar Harbor, ME) were subcutaneously inoculated into the right flank with 3×10⁶ multiple myeloma MM1S cells kindly provided by Steven Rosen at Northwestern University, Chicago, IL, USA in 100 μL RPMI 1640 medium and 100 μL of Matrigel (BD Biosciences). When tumours became palpable, mice were randomized to 8 groups of treatment with 5 mice in each one.

The groups were:
Control (group treated with vehicle alone)
Bortezomib 1 mg/kg twice weekly intraperitoneal for three weeks
Dexamethasone 0.5 mg twice weekly intravenously for three weeks
EDO-S101 intravenously at doses of 30 mg/kg once weekly for 3 doses,
Bortezomib plus dexamethasone
Bortezomib plus EDO-S101
EDO-S101 plus dexamethasone
Triple combination of EDO-S101 plus Bortezomib and dexamethasone

Caliper measurements of the tumor diameters were performed every day, and the tumor volume was estimated as the volume of an ellipse using the following formula: V=4/3π×(a/2)×(b/2)2, where “a” and “b” correspond to the longest and shortest diameter, respectively.

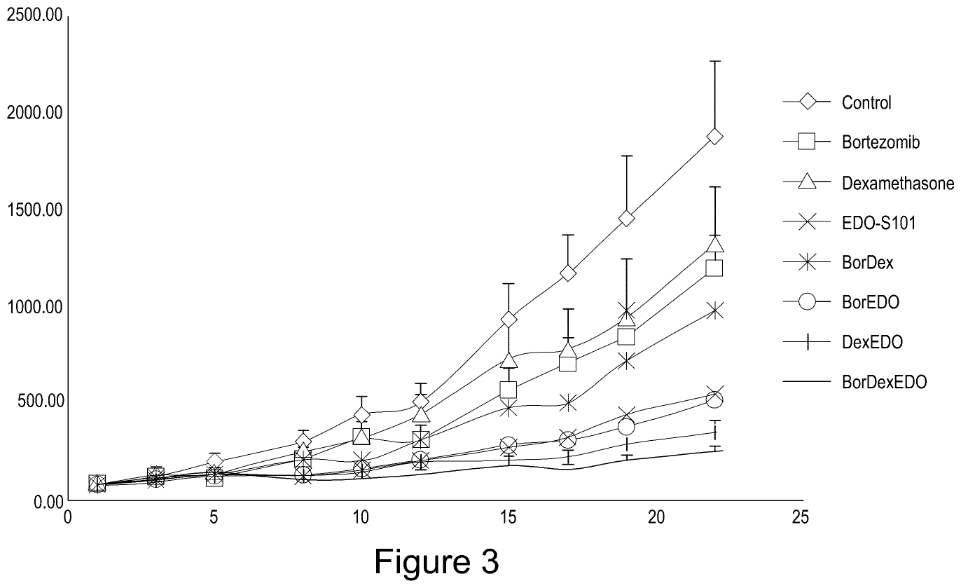

The tumour growth results are as shown in FIG. 3 in a plot of tumour growth (mm³) against the number of days of study. It can be seen that the combination of EDO-S101 and bortezomib results in tumour volumes lower than that seen with either agent alone while the triple combination of EDO-S101, bortezomib and dexamethasone shows very significantly lower tumour volumes by the end of the study than any of the active agents individually.

### Example 3 EDO-S101 Combinations In Vitro—Multiple Myeloma RPMI 8226 Cell Line

Using the same test procedure as described in Example 1, but using the multiple myeloma RPMI 8226 cell line (obtained from DMSZ) in place of the MM1S cell line, combinations of EDO-S101 with bortezomib, carfilzomib and LU-102 were tested for activity in turn. The concentrations for the different drugs were ratio constant for all the experiments. EDO-S101 at a concentration of 0, 2, 4, 8 μM; each of bortezomib and carfilzomib at a concentration of 0, 5, 10, 20 nM; and LU-102 at a concentration of 0, 1, 3.3, 10 μM. Controls with bendamustin were also performed.

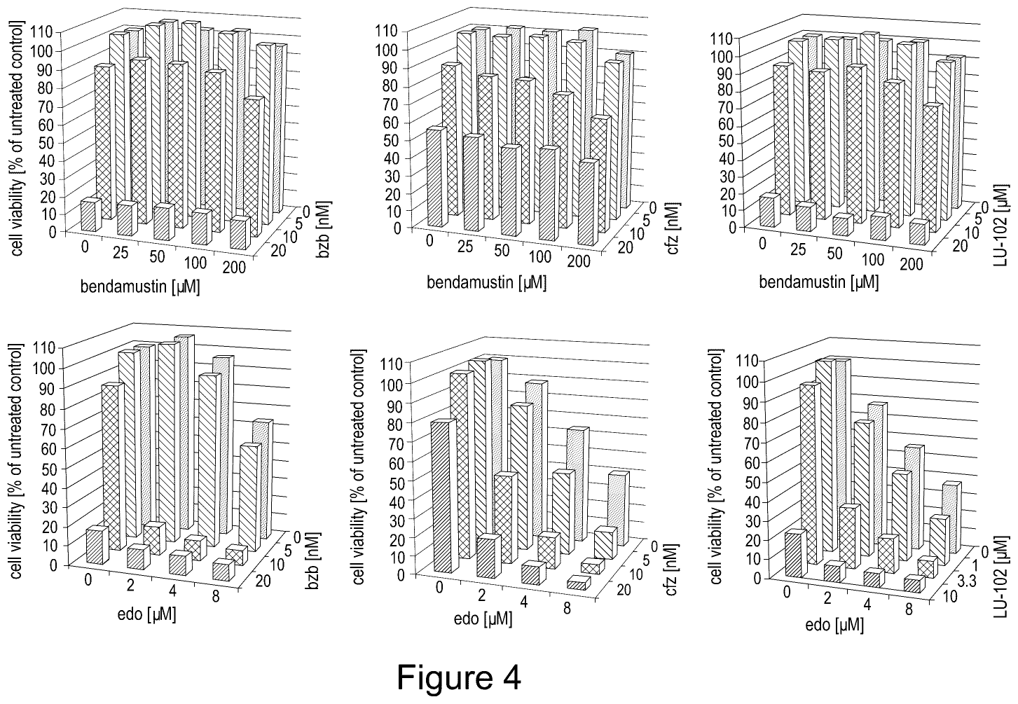

The cell viability as a percentage of the untreated control was measured and the results are as shown in FIG. 4. The figure shows clear synergy for each of the three combinations with EDO-S101 in vitro against multiple myeloma RPMI 8226. The CI at 4 μM EDO-S101 and 20 nm carfilzomib was 0.019 and the CI at 4 μM EDO-S101 and 3 μM LU-102 was 0.109.

### Example 4 EDO-S101 Combinations In Vitro—Multiple Myeloma Cell Line 2013 Oct. 16 MTS AMO abzb

Using the same test procedure as described in Example 1, but using the bortezomib resistant multiple myeloma 2013 Oct. 16 MTS AMO abzb cell line (generated at the Department of Oncology and Hematology of the Kantonsspital St. Gallen by Prof. Dr. med. C. Driessen) in place of the MM1S cell line, combinations of EDO-S101 with bortezomib, carfilzomib and LU-102 were tested for activity in turn. The concentrations for the different drugs were ratio constant and were 0, 2, 4, 8 μM for EDO-S101; 0, 1.25, 2.5, 5, 10, 20 nM for each of bortezomib and carfilzomib; and 0, 1, 3.3, 10 for LU-102.

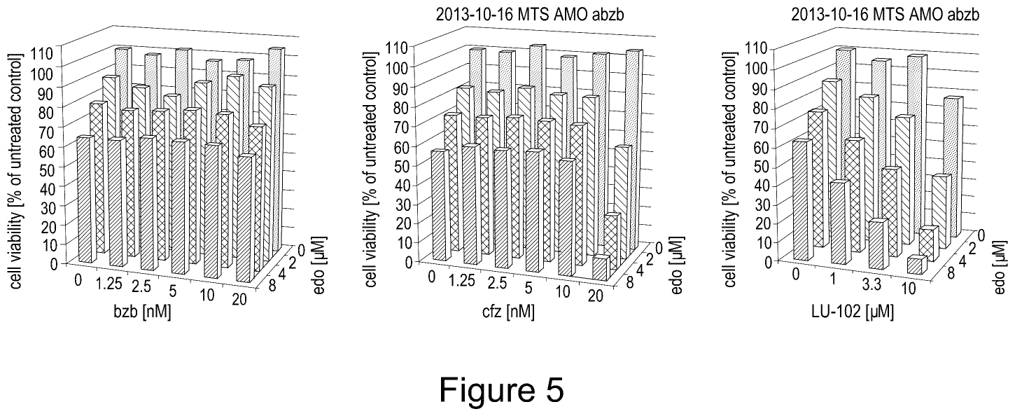

The cell viability as a percentage of the untreated control was measured and the results are as shown in FIG. 5. The figure shows clear synergy for the combinations of carflizomib and LU-102 with EDO-S101 in vitro against the bortezomib resistant multiple myeloma 2013 Oct. 16 MTS AMO abzb. The CI for the combinations of EDO-S101 and carfilzomib against this cell line was 0.11 and that for EDO-S101 and LU-102 was 0.25.

### Example 5 EDO-S101 Combinations In Vitro—Mantle Cell Lymphoma Cell Line 2014 Jan. 15 MTS Jeko

Using the same test procedure as described in Example 1, ut using the mantle cell lymphoma cell line 2014 Jan. 15 MTS Jeko (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and the same as in Example 3.

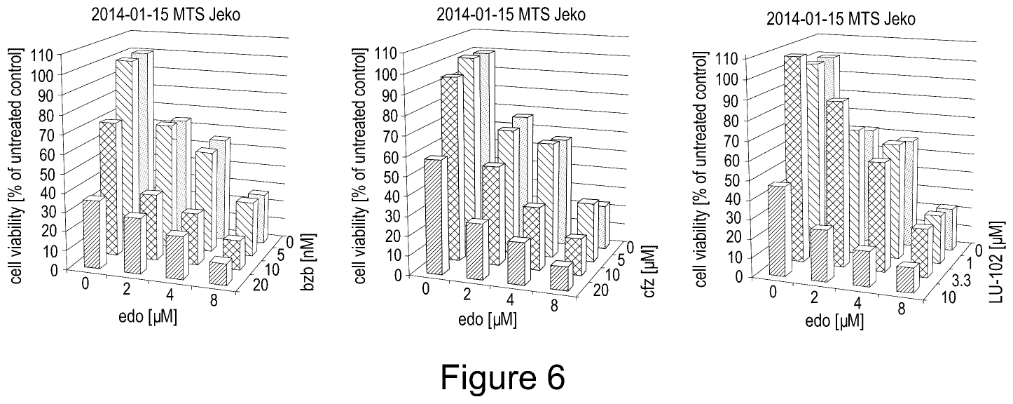

The cell viability as a percentage of the untreated control was measured and the results are as shown in FIG. 6. The figure shows clear synergy for each of the three combinations with EDO-S101 in vitro against mantle cell lymphoma cell line 2014 Jan. 15 MTS Jeko. The CI at 2 μM EDO-S101 and 20 nm bortezomib was 0.292; the CI at 2 μM EDO-S101 and 20 nm carfilzomib was 0.206; and the CI at 2 μM EDO-S101 and 10 μM LU-102 was 0.204.

### Example 6 EDO-S101 Combinations In Vitro—Mantle Cell Lymphoma Cell Line 2014 Jan. 15 MTS Granta

Using the same test procedure as described in Example 1, but using the mantle cell lymphoma cell line 2014 Jan. 15 MTS Granta (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and the same as in Example 3.

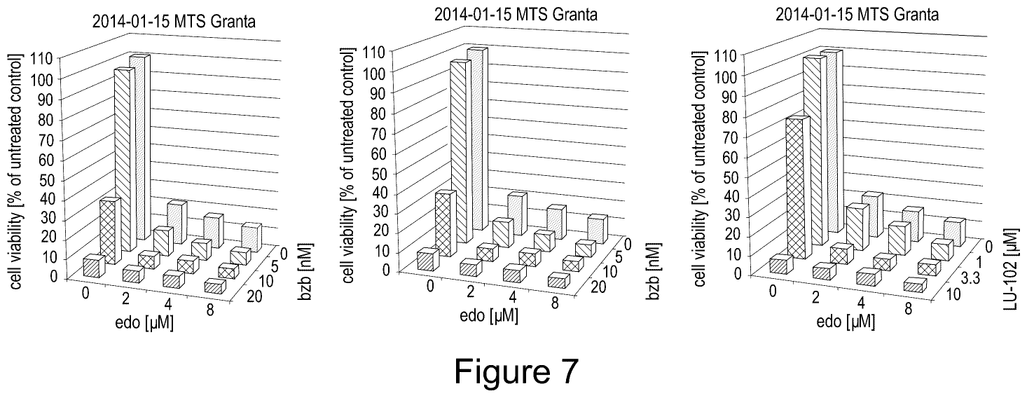

The cell viability as a percentage of the untreated control was measured and the results are as shown in FIG. 7. The figure shows clear synergy for each of the three combinations with EDO-S101 in vitro against mantle cell lymphoma cell line 2014 Jan. 15 MTS Granta. The CI at 0.5 μM EDO-S101 and 8 nm bortezomib was 0.025; the CI at 0.5 μM EDO-S101 and 8 nm carfilzomib was 0.089; and the CI at 1 μM EDO-S101 and 3 μM LU-102 was 0.078.

### Example 7 EDO-S101 Combinations In Vitro—Basal Like Breast Cancer Cell Line MTS MDA-MB468

Using the same test procedure as described in Example 1, but using the basal like breast cancer cell line MTS MDA-MB468 (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and were 0, 2, 4, 8 and 16 μM for EDO-S101; 0, 8, 16 and 32 nM for each of bortezomib and carfilzomib; and 0, 1, 3.3 and 10 μM for LU-102.

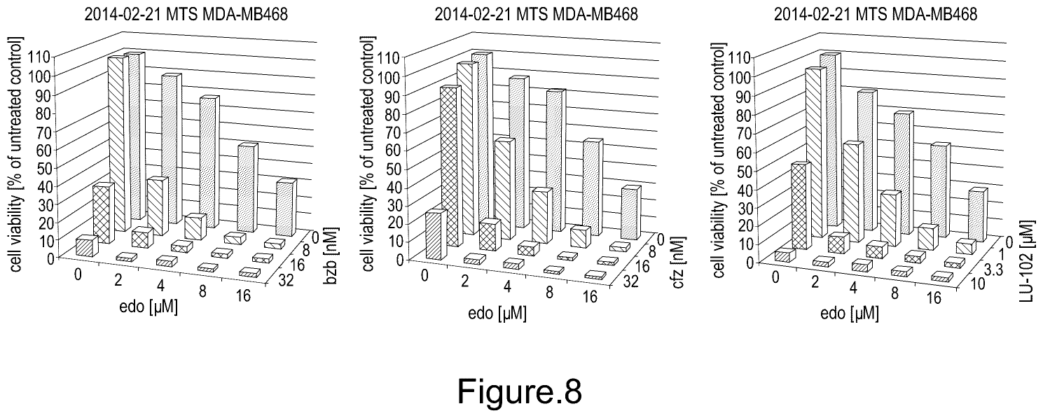

The cell viability as a percentage of the untreated control was measured and the results are as shown in FIG. 8. The figure shows clear synergy for each of the three combinations with EDO-S101 in vitro against this triple negative breast cancer cell line MTS MDA-MB468.

### Example 8 EDO-S101 Combinations In Vitro—Promyelocytic Leukemia Cell Line HL-60

Using the same test procedure as described in Example 1, but using the promyelocytic leukemia cell line HL-60 (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and were 0, 1, 2 and 4 μM for EDO-S101; 0, 5, 10, 20 nM for bortezomib and carfilzomib; and LU-102 for 0, 1, 3.3, 10 μM.

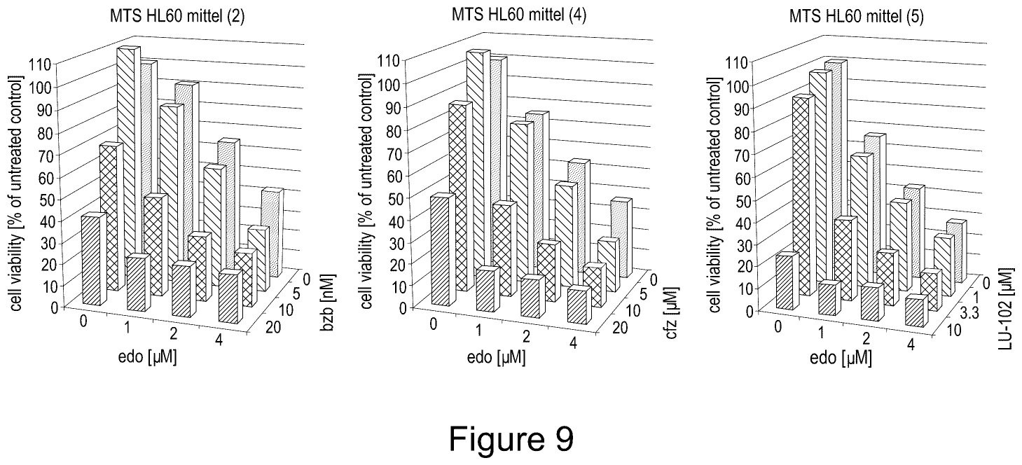

The cell viability as a percentage of the untreated control was measured and the results are as shown in FIG. 9. The figure shows clear synergy for each of the three combinations with EDO-S101 in vitro against promyelocytic leukemia cell line HL-60. The CI at 1 μM EDO-S101 and 20 nm bortezomibzomib was 0.051; the CI at 1 μM EDO-S101 and 20 nm carfilzomib was 0.073; and the CI at 1 μM EDO-S101 and 3 μM LU-102 was 0.387.

### Example 9 EDO-S101 Combinations In Vitro—Acute Myeloid Leukemia Cell Line U937

Using the same test procedure as described in Example 1, but using the acute myeloid leukemia cell line U937 (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and were the same as in Example 8.

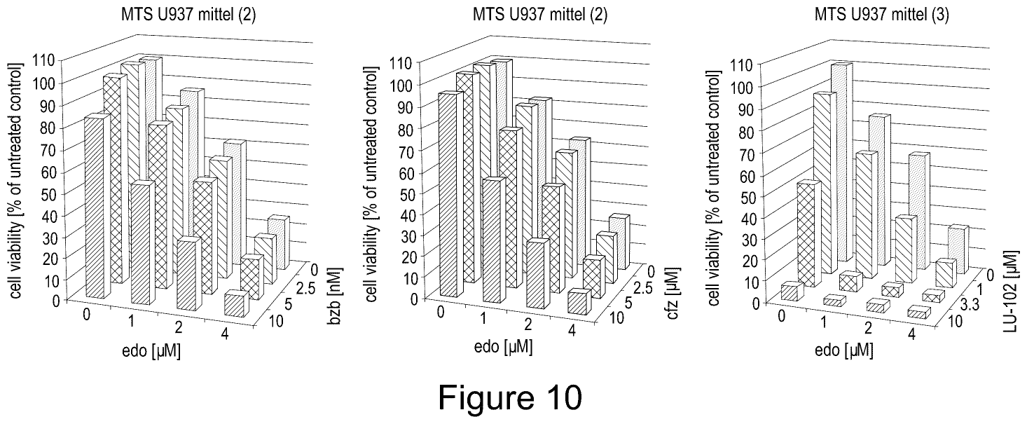

The cell viability as a percentage of the untreated control was measured and the results are as shown in FIG. 10. The figure shows clear synergy for each of the three combinations with EDO-S101 in vitro against basal like acute myeloid leukemia cell line U937. The CI at 2 μM EDO-S101 and 10 nm bortezomib was 0.285; the CI at 2 μM EDO-S101 and 10 nm carfilzomib was 0.272; and the CI at 2 μM EDO-S101 and 3 μM LU-102 was 0.095.

### Example 10 EDO-S101 Combinations In Vitro—B Cell Lymphoma Cell Line BJAB

Using the same test procedure as described in Example 1, but using the B cell lymphoma cell line BJAB (germinal center line) (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and were the same as in Example 8.

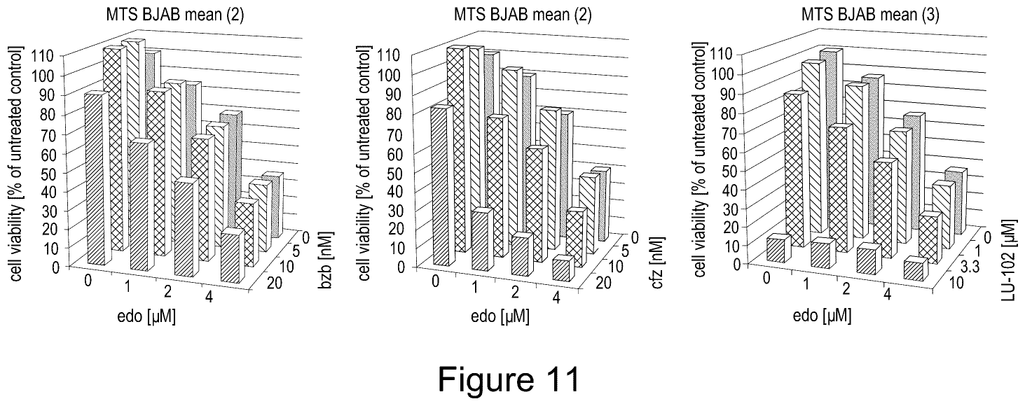

The cell viability as a percentage of the untreated control was measured and the results are as shown in FIG. 11. The figure shows strong synergy for the combination of EDO-S101 and carfilzomib in particular in vitro against B cell lymphoma cell line BJAB (germinal center line), while the combination of EDO-S101 and bortezomib also showed synergy. The CI for the combination of EDO-S101 and carfilzomib was 0.09, while the CI for the combination of EDO-S101 and bortezomib was 0.62.

### Example 11 EDO-S101 Combinations In Vitro—B Cell Lymphoma Cell Line OciLy3

Using the same test procedure as described in Example 1, but using the B cell lymphoma cell line OciLy3 (ABC-type) (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and were 0, 0.5, 1 and 2 μM for EDO-S101, 0, 5, 10 and 20 nM for bortezomib and carfilzomib and 0, 1, 3.3 and 10 μM for LU-102.

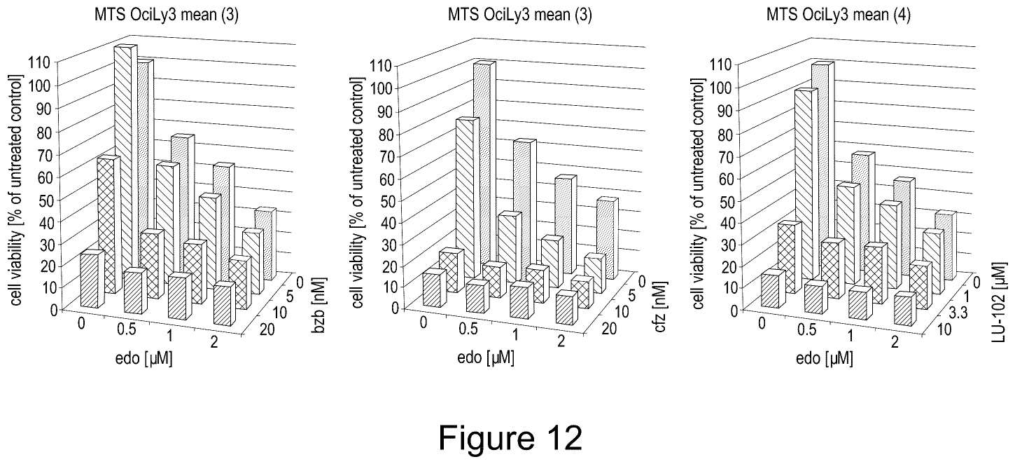

The cell viability as a percentage of the untreated control was measured and the results are as shown in FIG. 12. The figure shows strong synergy for the combination of EDO-S101 and bortezomib in particular in vitro against B cell lymphoma cell line OciLy3 (ABC-type), while the combination of EDO-S101 and carfilzomib also showed synergy. The CI for the combination of EDO-S101 and carfilzomib was 0.59, while the CI for the combination of EDO-S101 and bortezomib was 0.21.

### Example 12 EDO-S101 Combinations In Vitro—B Cell Lymphoma Cell Line TMD8

Using the same test procedure as described in Example 1, but using the B cell lymphoma cell line TMD8 (ABC-type) (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and were the same as in Example 11.

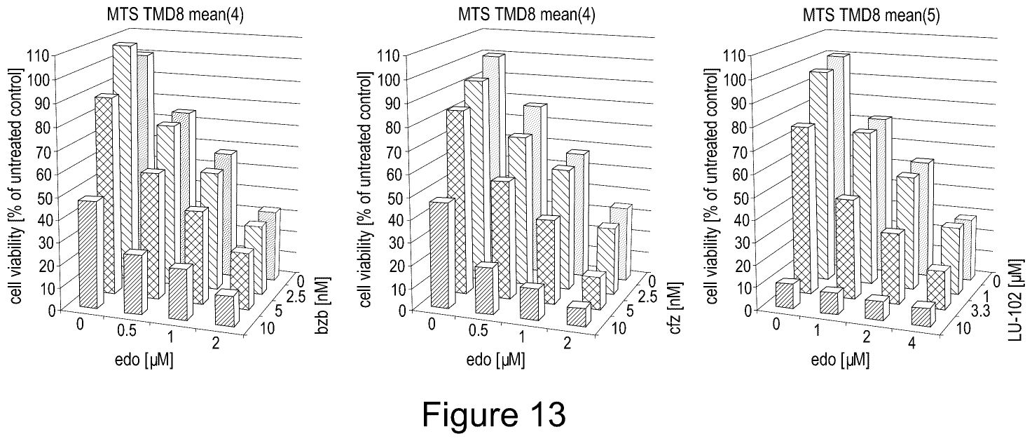

The cell viability as a percentage of the untreated control was measured and the results are as shown in FIG. 13. The figure shows strong synergy for all combinations of EDO-S101 and proteasome inhibitor tested. The CI for the combination of EDO-S101 and carfilzomib was 0.17, the CI for the combination of EDO-S101 and bortezomib was 0.14 and the CI for the combination of EDO-S101 and LU-102 was 0.63.

### Example 13 EDO-S101 Combinations In Vitro—Triple Negative Breast Cancer Cell Line BT-549

Using the same test procedure as described in Example 1, but using the triple negative breast cancer cell line BT-549 (obtained from LGC Standards S.a.r.l., 6, rue Alfred Kastler, BP 83076, F-67123 Molsheim Cedex, France) in place of the MM1S cell line, combinations of EDO-S101 with bortezomib, carfilzomib and LU-102 were tested for activity in turn. The concentrations for the different drugs were ratio constant for all the experiments and were 0, 1, 2 and 4 μM for EDO-S101; 0, 5, 10 and 20 nM for each of bortezomib and carfilzomib; and 0, 1, 3.3 and 10 μM for LU-102.

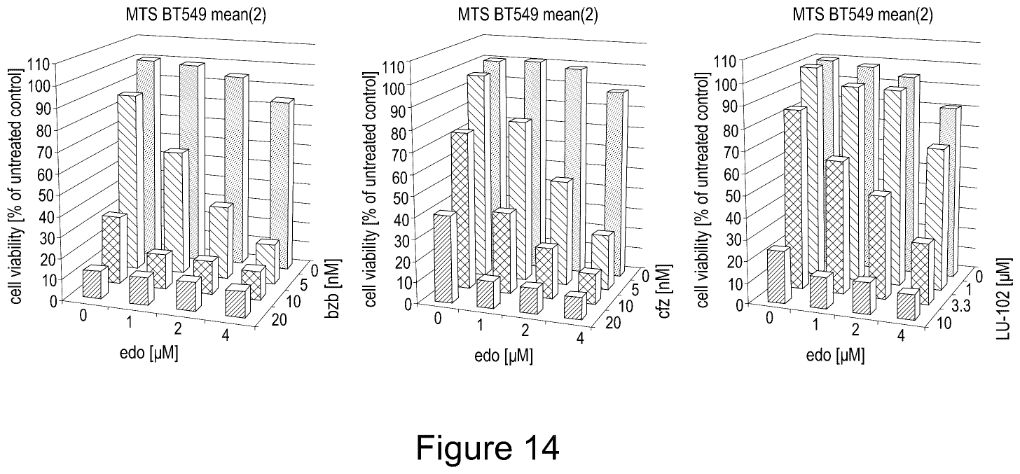

The cell viability as a percentage of the untreated control was measured and the results are as shown in FIG. 14. The figure shows clear synergy for each of the three combinations with EDO-S101 in vitro against triple negative breast cancer cell line BT-549. The CI for the combination of EDO-S101 and bortezomib was 0.14, the CI for the combination of EDO-S101 and carfilzomib was 0.05 and the CI for the combination of EDO-S101 and LU-102 was 0.38.

### Example 14 Combinations of Radiotherapy and EDO-S101 Against Glioblastoma Cell Lines In Vitro

For the U251MG glioblastoma cell line, the IC₅₀ was measured to be 6.60 μM for EDO-S101 (compared to 30 μM for bendamustin and 20 for temozolamide).

For the U87G glioblastoma cell line, the IC₅₀ was measured to be 1.36 μM for EDO-S101 (compared to 50 μM for bendamustin and 20 for temozolamide).

For the T98G glioblastoma cell line, the IC₅₀ was measured to be 7.70 μM for EDO-S101 (compared to 52 82 M for bendamustin and >100 for temozolamide).

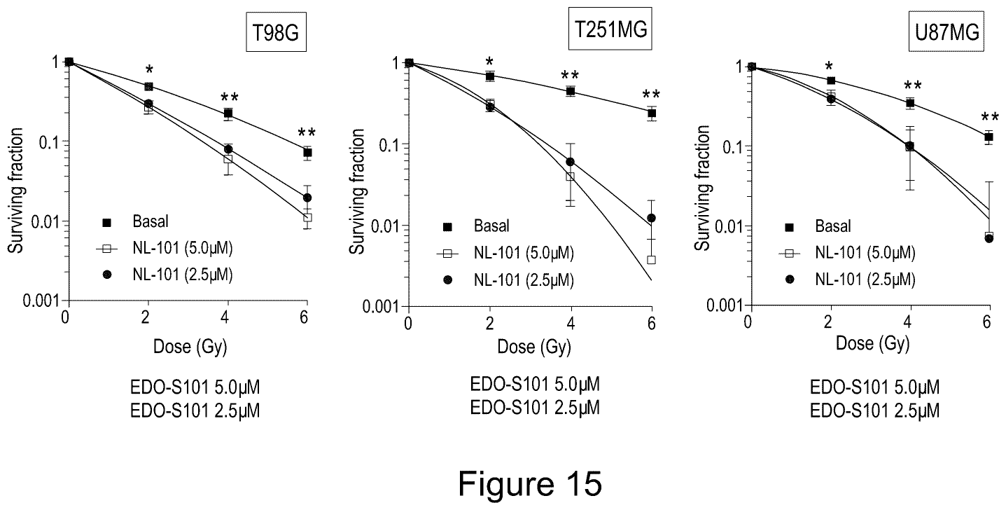

As can be seen from FIG. 15, the % survival rate for the glioblstoma cells was considerably reduced when radiotherapy was used in combination with a dose of EDO-S101 (5 μM or 10 μM) compared to radiotherapy alone.

### Example 15 Combinations of Radiotherapy and EDO-S101 Against Glioblastoma Cell Lines In Vivo

U87MG, U251MG and T98G

### Subcutaneously Inoculated Xenografts

### Treatments and Doses

Vehicle (control)
Radiotherapy (2Gy/5 consecutive days)
Temozolamide (16 mg/Kg for 5 consecutive days, po)
Temozolamide+radiotherapy
EDO-S101 (60 mg/Kg at day 1, 8 and 15 every 28 days, iv)
EDO-S101+radiotherapy

It was found that the time to progression of the tumours was increased from approximately 17-18 days for the control for the U251MG mouse xenograft model, to 42 days with a combination of radiotherapy and temozolamide to over 50 days for EDO-S101 alone (significance P=0.924) to significantly over 50 days for a combination of EDO-S101 and radiotherapy (significance P=0.0359).

It was found that the time to progression of the tumours was increased from approximately 15 days for the control for the U87MG mouse xenograft model, to 35 days with a combination of radiotherapy and temozolamide to 40 days for EDO-S101 alone (significance P=2372) to significantly over 50 days for a combination of EDO-S101 and radiotherapy (significance P=0.0001).

### Example 16 Activity of EDO-S101 Against Relapsed/Refractory Multiple Myeloma Models

A genetic rearrangement of the MYC locus, resulting in dysregulated expression of MYC, is the most common mutation in human multiple myeloma. The genetically engineered Vk*MYC mouse model is based on dysregulation of MYC, and has been extensively validated as a clinically and biologically faithful model of untreated multiple myeloma. Nine drugs or classes of drugs (DNA alkylators, glucocorticoids, proteasome inhibitors, IMiDs, nab-paclitaxel, histone deacetylase inhibitors, TACI-Ig, perifosine and SNS-032, a CDK2, 7, 9 inhibitor) have been previously reported with more than a 20% partial response rate in Vk*MYC MM. Among those, the first five also have greater than 20% PR in patients with multiple myeloma for a positive predictive value of 56%.

EDO-S101 induced a high rate of response in Vk*MYC multiple myeloma that was sustained for more than three months in mice receiving only two doses, one week apart. Remarkably EDO-S101 is the only drug that was identified with single agent activity in the very aggressive, multi-drug resistant Vk12653 transplant model of relapsed/refractory multiple myeloma.

In conclusion, it can be seen that the compound of formula I (EDO-S101) show excellent activity in combination with proteasome inhibitors in acting both in vitro and in vivo against a wide range of myeloma, lymphoma, leukemia and breast cell lines. Furthermore, it can be seen that the activity of many of these combinations is surprisingly synergistic, and in many cases to a very significant degree. Yet further, it is seen in Examples 1 and 2 that triple combinations comprising the compound of formula I, a proteasome inhibitor and a glucocorticoid such as dexamethasone showed particularly strong synergy.

As a result, it is to be expected that combinations of the compound of formula I of the present invention with a proteasome inhibitor, optionally comprising a glucocorticoid, will be of use in the treatment of cancer, particularly hematologic cancers and breast cancer.

1-42. (canceled)

43. A method of treating glioblastoma in a patient in need thereof comprising administering to said patient an effective amount of a compound of formula I or a pharmaceutically acceptable salt thereof: 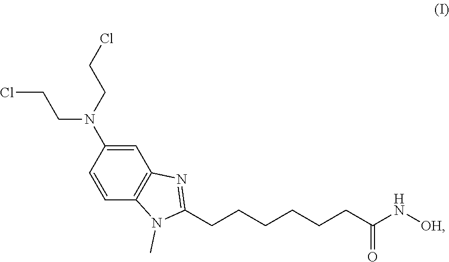 [SMILES: Cn1c(CCCCCCC(=O)NO)nc2cc(N(CCCl)CCCl)ccc21]
wherein the patient is also being treated with radiotherapy.

44. The method according to claim 43, wherein the method comprises treating the patient for at least a treatment period of 28 days.

45. The method of claim 44, wherein the radiotherapy is administered at a dose of 2 Gy.

46. The method of claim 45, wherein the radiotherapy is administered for 5 consecutive days.

47. The method according to claim 44, wherein the compound of formula I or pharmaceutically acceptable salt thereof is administered to the patient at day 1, 8, and 15 of each treatment period.

48. The method of claim 43, wherein the compound of formula I or pharmaceutically acceptable salt thereof is administered intravenously to the patient.

49. The method of claim 43, wherein the compound of formula I or pharmaceutically acceptable salt thereof is administered orally to the patient.

50. The method of claim 43, wherein the effective amount of the compound of formula I or pharmaceutically acceptable salt thereof is from 10 to 100 mg/kg body weight of the patient.

51. The method of claim 50, wherein the effective amount of the compound of formula I or pharmaceutically acceptable salt thereof is 60 mg/kg body weight of the patient.
